(19) United States
(12) Patent Application Publication
(10) Pub. No.: US 2026/0131021 A1
(43) Pub. Date: May 14, 2026

(54) LIPID NANOPARTICLE-BASED DRUG DELIVERY SYSTEM USING RECOMBINANT PROTAMINE AND METHOD FOR PREPARING SAME

(71) Applicant: MOOGENE MEDI CO., LTD., Hanam-si, Gyeonggi-do (KR)

(72) Inventors: Tae Jong YOON, Seoul (KR); Eun Jeong WON, Incheon (KR)

(73) Assignee: MOOGENE MEDI CO., LTD., Hanam-si, Gyeonggi-do (KR)

(21) Appl. No.: 18/852,867

(22) PCT Filed: Jan. 12, 2023
(86) PCT No.: PCT/KR2023/000559

§ 371 Date: Jun. 26, 2025

(30) Foreign Application Priority Data
Apr. 22, 2022 (KR) 10-2022-0050206
Jun. 28, 2022 (KR) 10-2022-0078598

(51) Int. Cl. A61K 48/00; A61K 47/42; C07K 14/47

(52) U.S. Cl. CPC A61K 48/0033; A61K 47/42; C07K 14/47

(57) ABSTRACT

The present invention relates to a lipid nanoparticle-based drug delivery system using recombinant protamine and a method for preparing same. The lipid nanoparticle-based drug delivery system of the present invention can stably deliver a gene to a target cell in vivo by using a recombinant protamine protein having excellent stability when forming a complex with an anionic gene. In addition, the lipid nanoparticle-based drug delivery system can improve the expression level of the gene in the target cell, and thus can be effectively used in related technical fields such as lipid nanoparticle-mediated gene therapy.

### TECHNICAL FIELD

The present disclosure relates to a lipid nanoparticle-based drug delivery system using recombinant protamine and to a method of preparing the same.

### BACKGROUND ART

In the pharmaceutical preparation industry, a drug delivery system (DDS) is designed to efficiently deliver the required amount of drugs while reducing the side effects of drugs and maximizing their efficacy and effectiveness. The system is a high-value-added core technique since the system may generate economic benefits equivalent to the development of new drugs and has a high potential for success. Therefore, the purpose of the drug delivery system (DDS) is to improve the quality of patient treatment by streamlining drug administration.

As one of the core techniques of the drug delivery system, there is a solubilization technique for poorly soluble drugs, which belongs to a drug absorption promotion technique. This solubilization technique is considered the most reasonable way to reduce the development cost of new drug substances and at the same time to increase the added value of currently available drugs. Especially in situations where new drug development conditions are poor, such as in Korea, developing improved new drugs with a developed drug solubilization technique may create enormous added value at a low cost.

Meanwhile, gene therapy using genetic drug delivery systems holds great hope in modifying genetic deficits and treating numerous diseases. Effective gene delivery is a major challenge in gene therapy's successful and safe implementation. So far, viral vectors have been proven to be effective in gene transfer. However, there are several problems with the viral vectors such as immunogenicity, tumorigenic potential, cytotoxicity, limited DNA size in the viral vector and complex production process, and limitation of repeated administration due to immune response. Accordingly, the use of viruses as a gene delivery system is limited. As an alternative to viral systems, non-viral gene carriers such as cationic liposomes and polymers that may overcome the problems have begun to attract attention.

To become an ideal non-viral gene carrier, genetic material is required to be condensed to form a nano-sized complex to be absorbed by cells. In addition, the carrier is required to maintain a stable structure to facilitate delivery to cells and to keep its stable form from enzymatic reaction-caused decomposition in the body until external genetic material is delivered to the cell nucleus. To prepare an ideal virus gene carrier described above, attempts have been made to form a gene complex using only cationic polymers, proteins, or peptides, or to use cationic lipid nanoparticles. For example, as a method to facilitate the delivery of nucleic acids within cells or the body, it has been reported that nucleic acid-lipid particles containing conjugated lipids that inhibit aggregation of nucleic acids, cationic lipids, non-cationic lipids, and particles could be adopted (Patent Document 0001). In addition, it has been reported that lipid nanoparticles can be adopted as carriers to efficiently deliver nucleic acids such as siRNA into target cells by taking pH-sensitive cationic lipids as constituent lipids of the nanoparticles (Patent Document 0002).

Protamine, a type of cationic peptide used among researchers for non-viral gene delivery, is a natural DNA condensing agent. Protamine is known for its unique role in condensing DNA to form the dense structure of sperm and delivering sperm DNA to the egg nucleus after fertilization. Protamine sulfate (PS), obtained from salmon testicles, is mainly used as protamine. PS has a poly-arginine structure, so the positive charge ratio of PS is very high. Therefore, researchers frequently use PS as a non-viral gene delivery system. However, commercialized PA has poor aqueous solution stability when complexed with anionic genes. The poor stability of PA is shown even when PA is encapsulated in lipid nanoparticles.

Under this technical background, the present inventors have diligently developed a drug delivery system capable of efficiently delivering anionic drugs and nucleic acids to target organs or cells. As a result, a recombinant protamine protein with improved stability when forming a complex with nucleic acid was prepared. By confirming that lipid nanoparticles encapsulating a protamine-nucleic acid complex of a recombinant protamine protein and a nucleic acid showed excellent stability and allowed for significantly high gene expression levels at the cellular level, the present disclosure was completed.

### RELATED ART DOCUMENTS

### Patent Documents

(Patent Document 0001) U.S. Pat. No. 9,404,127 (Registration Date: 2016 Aug. 2)
(Patent Document 0002) Korean Patent Application Publication No. 10-2020-0018782 (Publication date: 2020 Feb. 20)

### DISCLOSURE

### Technical Problem

The present disclosure is to provide a lipid nanoparticle-based drug system containing a recombinant protamine protein, which includes a solubility enhancing tag and protamine, and to provide a method of preparing the same to stably deliver anionic drugs such as nucleic acids into organs or cells.

### Technical Solution

To achieve the objective,
the present disclosure provides a lipid nanoparticle-based drug delivery system including: (a) a protamine-nucleic acid complex of a recombinant protamine protein and a nucleic acid, the recombinant protamine protein including a solubility enhancing tag and a protamine made of an amino acid having a sequence of SEQ ID NO: 1;
(b) a non-cationic lipid; and
(c) a PEG-modified lipid.

In addition, the present disclosure provides a method of preparing a lipid nanoparticle-based drug delivery system, the method including: (a) preparing a protamine-nucleic acid complex by mixing a nucleic acid solution and a solution of a recombinant protamine protein including a solubility enhancing tag and a protamine made of an amino acid having a sequence of SEQ ID NO:
(b) forming a lipid film by introducing a lipid solution including an organic solvent, a non-cationic lipid, and a PEG-modified lipid into a reactor and removing the organic solvent; and
(c) forming a lipid nanoparticle dispersion liquid by applying and dispersing the complex prepared in the step (a) onto the lipid film formed in the step (b).

### Advantageous Effects

The lipid nanoparticle-based drug delivery system of the present disclosure can stably deliver genes to target cells in the body by using a recombinant protamine protein that shows excellent stability when forming a complex with an anionic gene. In addition, the lipid nanoparticle-based drug delivery system can improve gene expression levels within target cells. Thus, the lipid nanoparticle-based drug delivery system can be usefully applied in related technical fields such as lipid nanoparticle-mediated gene therapy.

### DESCRIPTION OF DRAWINGS

FIG. 1A illustrates a diagram showing the structure of a lipid nanoparticle encapsulating a protamine-nucleic acid complex of a nucleic acid and a recombinant protamine protein made of an amino acid having a sequence of SEQ ID NO: 1;

FIG. 1B illustrates a schematic diagram showing the synthesis process of a lipid nanoparticle;

FIG. 2A illustrates a diagram showing the results of cloning a recombinant protamine protein-encoding gene sequence into an E. coli expression plasmid (pET30a-PA);

FIG. 2B illustrates a diagram showing the purity of the recombinant protamine protein in each step of a purification process;

FIG. 2C illustrates a diagram confirming whether there was E. coli RNA contamination during the purification process of the recombinant protamine protein;

FIG. 3 illustrates a diagram confirming the optimal concentration to form a complex of a recombinant protamine protein and a plasmid DNA through agarose gel electrophoresis analysis;

FIG. 4A illustrates a graph showing the results of comparing the stability when forming a complex by mixing 30 μM of recombinant protamine protein or 30 μM of existing protamine sulfate with 7.5 nM of plasmid DNA, respectively, using a UV-visible spectrometer;

FIG. 4B illustrates a graph showing the results of comparing the stability when forming a complex by mixing 30 μM of recombinant protamine protein or 30 μM of existing protamine sulfate with 15 nM of plasmid DNA, respectively, using a UV-visible spectrometer;

FIG. 4C illustrates a graph showing the results of comparing the stability when forming a complex by mixing 30 μM of recombinant protamine protein or 30 μM of existing protamine sulfate with 30 nM of plasmid DNA, respectively, using a UV-visible spectrometer;

FIG. 4D illustrates a diagram visually confirming the stability when 30 μM of the recombinant protamine protein was mixed with plasmid DNA at its respective concentrations of 0, 7.5, 15, 30, and 60 nM to form a complex;

FIG. 4E illustrates a diagram visually confirming the stability when 30 μM of existing protamine sulfate was mixed with plasmid DNA at its respective concentrations of 0, 7.5, 15, 30, and 60 nM to form a complex;

FIG. 5A illustrates a diagram visually confirming the stability of lipid nanoparticles when stored at 4° C. after lipid nanoparticles had been synthesized using each complex formed by mixing 30 μM of existing protamine sulfate with plasmid DNA at its respective concentrations of 0, 7.5, 15, 30, and 60 nM;

FIG. 5B illustrates a diagram visually confirming the stability of lipid nanoparticles when stored at 4° C. after lipid nanoparticles had been synthesized using each complex formed by mixing 30 μM of recombinant protamine protein with plasmid DNA at its respective concentrations of 0, 7.5, 15, 30, and 60 nM;

FIG. 5C illustrates a graph showing the results of dynamic light scattering (DLS) analysis to confirm a sample size after 24 hours at 4° C., after lipid nanoparticles had been synthesized using 30 UM of existing protamine sulfate in the absence of plasmid DNA;

FIG. 5D illustrates a graph showing the results of dynamic light scattering (DLS) analysis to confirm a sample size after 24 hours at 4° C., after lipid nanoparticles had been synthesized using each complex formed by mixing 30 μM of existing protamine sulfate with 7.5 nM of plasmid DNA;

FIG. 5E illustrates a graph showing the results of dynamic light scattering (DLS) analysis to confirm a sample size after 24 hours at 4° C., after lipid nanoparticles had been synthesized using each complex formed by mixing 30 μM of existing protamine sulfate with 15 nM of plasmid DNA;

FIG. 5F illustrates a graph showing the results of dynamic light scattering (DLS) analysis to confirm a sample size after 24 hours at 4° C., after lipid nanoparticles had been synthesized using each complex formed by mixing 30 μM of existing protamine sulfate with 30 nM of plasmid DNA;

FIG. 5G illustrates a graph showing the results of dynamic light scattering (DLS) analysis to confirm a sample size after 24 hours at 4° C., after lipid nanoparticles had been synthesized using each complex formed by mixing 30 μM of existing protamine sulfate with 60 nM of plasmid DNA;

FIG. 5H illustrates a graph showing the results of dynamic light scattering (DLS) analysis to confirm a sample size after 24 hours at 4° C., after lipid nanoparticles had been synthesized using 30 μM of recombinant protamine protein in the absence of plasmid DNA;

FIG. 5I illustrates a graph showing the results of dynamic light scattering (DLS) analysis to confirm a sample size after 24 hours at 4° C., after lipid nanoparticles had been synthesized using each complex formed by mixing 30 μM of recombinant protamine protein with 7.5 nM of plasmid DNA;

FIG. 5J illustrates a graph showing the results of dynamic light scattering (DLS) analysis to confirm a sample size after 24 hours at 4° C., after lipid nanoparticles had been synthesized using each complex formed by mixing 30 μM of recombinant protamine protein with 15 nM of plasmid DNA;

FIG. 5K illustrates a graph showing the results of dynamic light scattering (DLS) analysis to confirm a sample size after 24 hours at 4° C., after lipid nanoparticles had been synthesized using each complex formed by mixing 30 μM of recombinant protamine protein with 30 nM of plasmid DNA;

FIG. 5L illustrates a graph showing the results of dynamic light scattering (DLS) analysis to confirm a sample size after 24 hours at 4° C., after lipid nanoparticles had been synthesized using each complex formed by mixing 30 μM of recombinant protamine protein with 60 nM of plasmid DNA; and

FIG. 6 illustrates a diagram confirming the protein expression level in the cells using Western blotting 24 hours after MDA-MB-468 cells were treated with lipid nanoparticles encapsulating a complex of 30 μM of recombinant protamine protein and 30.82 nM of plasmid DNA encoding HIC1 gene.

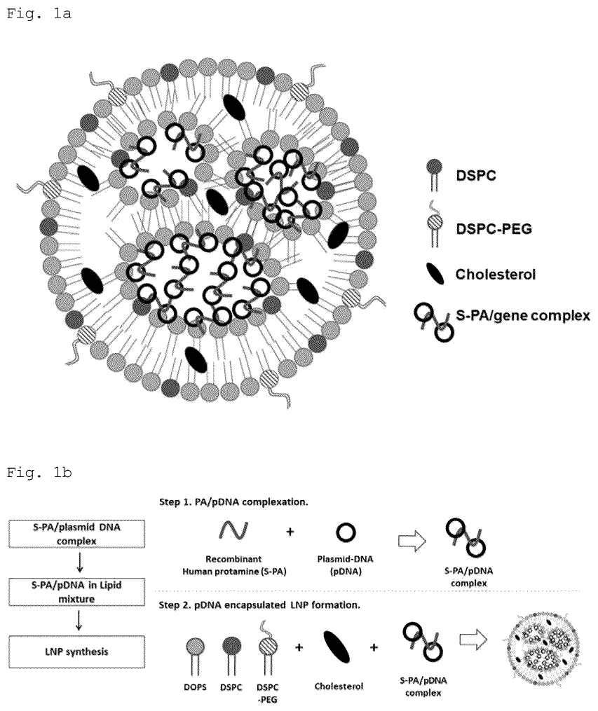

### MODE FOR DISCLOSURE

Hereinafter, a lipid nanoparticle-based drug delivery system using a recombinant protamine and a method of preparing the same according to the present disclosure will be described in detail.

Terms and words used in this specification and claims should not be construed as limited to their ordinary or dictionary meanings. Based on the principle that the inventor(s) may appropriately define the concept of the term to explain his or her invention in the best way, the terms and words are required to be interpreted as having meanings and concepts consistent with the technical idea of the present disclosure.

The present disclosure provides a lipid nanoparticle-based drug delivery system including: (a) a protamine-nucleic acid complex of a recombinant protamine protein and a nucleic acid, protamine protein including a solubility the recombinant enhancing tag and a protamine made of an amino acid having a sequence of SEQ ID NO: 1;
(b) a non-cationic lipid; and
(c) a PEG-modified lipid.

The protamine (SEQ ID NO: 1) is a protein expressed in cells transfected with a recombinant expression vector containing the protamine gene (SEQ ID NO: 2). The protamine is cationic and thus interacts electrostatically with therapeutic agents such as nucleic acids in a negative charge. Through electrostatic interaction, the protamine easily forms a complex with the drug, making it possible for the protamine to be highly efficiently encapsulated in lipid nanoparticles. The lipid nanoparticles encapsulating the complex may be useful as an intracellular or an in-vivo drug delivery vehicle for drugs (for example, nucleic acids).

As used herein, the term “encapsulation” refers to encapsulating a delivery material for efficient incorporation into the body.

The term “solubility enhancing tag” refers to a tag that may improve the aqueous solution stability of the complex when the protamine protein binds to an anionic nucleic acid to form a complex. The solubility enhancing tag may be used without limitation in its type as long as it may help the recombinant protamine protein to be expressed in a water-soluble form. For example, the solubility enhancing tag may include any one selected from the group consisting of S-tag, b′a′ domain of PDI (PDIb′a′), protein disulfide isomerase, maltose binding protein (MBP), hexahistidine (His6), thioredoxin (Trx), glutathione S-transferase (GST), N-utilization substance Protein A (NusA), small ubiquitin related modifier (SUMO), and protein disulfide isomerase (PDI).

The solubility enhancing tag may be bound to the N-terminus or C-terminus of the protamine, or bound to both the N-terminus and C-terminus of the protamine and one or more than two solubility enhancing tags may be bound sequentially.

In an embodiment of the present disclosure, the solubility enhancing tag may be the S-tag. The solubility enhancing tag may bind to the N-terminus of the protamine to form a recombinant protamine protein (S-PA, SEQ ID NO: 3). When the recombinant protamine protein bound with the S-tag combines with a nucleic acid to form a complex, the stability of the complex in an aqueous solution may be improved. Even when the complex is encapsulated, the stability of the lipid nanoparticles may be increased. Through this, the nucleic acid may be stably delivered into target cells, resulting in increased gene expression.

TABLE 1

| | | Sequence |
|---|---|---|
| Name | Sequence | Number |
| Protamine | ARYRCCRSQSRSRYYRQRQRSRRRRRRSCQTRRRAMRCCRPRYRPRCRRH | 1 |
| (amino acid | | |
| sequence) | | |
| Protamine gene | ATGGCGCGTTATCGTTGCTGCCGTAGCCAAAGCCGTAGCCGTTATTATCGT | 2 |
| (coding nucleic | CAGCGTCAGCGTAGCCGTCGTCGTCGTCGTCGTAGCTGCCAGACCCGCCGC | |
| acid sequence) | CGTGCGATGCGTTGCTGCCGTCCGCGTTACCGCCCGCGTTGCCGTCGTCAC | |
| S-tag bound | KETAAAKFERQHMDSARYRCCRSQSRSRYYRQRQRSRRRRRRSCQTRRRAM | 3 |
| protamine | RCCRPRYRPRCRRH | |
| (amino acid | | |
| sequence) | | |

The nucleic acid is a therapeutic agent for intracellular or in-vivo delivery and may form a complex with a cationic protamine through electrostatic interaction. For example, the nucleic acid may include one or more selected from the group consisting of plasmid DNA, single-stranded DNA, double-stranded DNA, miRNA, siRNA, ssRNA, shRNA, mRNA, ncRNA, antisense RNA, and LNA but is not limited thereto.

In this specification, the term “microRNA (miRNA)” refers to a single-stranded RNA molecule made of 21 to 23 nucleotides in total length. The miRNA regulates gene expression. The miRNA is an oligonucleotide, and the miRNA is not expressed within cells and has a short stem-loop structure. The miRNA has full or partial homology to one or two or more messenger RNAs (mRNAs). The miRNA suppresses target gene expression through complementary binding to the mRNAs.

In this specification, the term “siRNA” refers to a specific double-stranded RNA (duplex RNA), or a single-stranded RNA. At this point in the case of a single-stranded RNA form, the siRNA has a double-stranded form therein. The siRNA may induce RNA interference (RNAi) phenomenon through cleavage of specific mRNA. The siRNA is made of a sense RNA strand with a sequence homologous to the mRNA of the target gene and an antisense RNA strand with a complementary sequence. Since the siRNA may suppress the expression of the target gene, the siRNA is provided for an efficient gene knock-down method or a method of gene therapy. Bonding between double strands is achieved through hydrogen bonds between nucleotides. Not all nucleotides within the double strand are required to be completely bonded in a complementary manner.

The siRNA may have a length of about 15 to 60, specifically about 15 to 50, about 15 to 40, about 15 to 30, about 15 to 25, about 16 to 25, about 19 to 25, about 20 to 25, or about 20 to 23 nucleotides. The siRNA length refers to the number of nucleotides on one side of double-stranded RNA, that is, the number of base pairs. In the case of single-stranded siRNA, the length means that of the double-stranded region within the single-stranded RNA. Alternatively, the siRNA may have nucleotides with various functional groups for purposes such as increasing blood stability or weakening immune responses.

In this specification, the term “ssDNA” refers to a single-stranded oligonucleotide. The ssDNA selectively binds to specific target DNA and induces an antigenic effect.

In this specification, the term “shRNA” refers to a single strand made of 50 to 70 nucleotides, having a stem-loop structure in vivo. Long RNAs of 19 to 29 nucleotides complementarily base pair on both sides of the loop region of 5 to 10 nucleotides to form a double-stranded stem.

In this specification, the term “mRNA” refers to synthetic mRNA (in-vitro transcribed mRNA) capable of expressing genes.

In this specification, the term “ncRNA” is not translated into protein but functions as RNA itself. The ncRNA includes everything from tRNA and rRNA, which is commonly known, to snoRNA, miRNA, and lncRNA. The ncRNA is involved in transcription, splicing, and translation throughout the cell.

In this specification, the term “anti-sense RNA” is RNA or derivatives thereof containing a nucleic acid sequence complementary to the sequence of a specific mRNA. The antisense RNA binds to the complementary sequence within the mRNA and inhibits mRNA translation into protein. The antisense RNA refers to an RNA sequence complementary to and capable of binding to the mRNA of the genes. The antisense RNA may inhibit translation of the gene mRNA, translocation into the cytoplasm, maturation, or any other essential activity for overall biological function. The antisense RNA may have a length of 6 to 100 bases, specifically 8 to 60 bases, and more specifically 10 to 40 bases. The antisense RNA may be synthesized in vitro using a conventional method and administered in vivo, or the antisense RNA may be synthesized in vivo. One example of synthesizing antisense oligonucleotides in vitro is to use an RNA polymerase I. One example of allowing antisense RNA to be synthesized in vivo is to transcribe the antisense RNA using a vector with the origin of the multiple cloning site (MCS) in the opposite direction. It is preferable that the antisense RNA has a translation stop codon within the sequence so that it is not translated into a peptide sequence.

In this specification, the term “locked nucleic acid (LNA)” refers to nucleic acid analogs containing 2′-O, 4′-C methylene bridges [J Weiler, J Hunziker and J Hall Gene Therapy (2006) 13, 496.502]. LNA nucleosides contain the common nucleic acid bases of DNA and RNA, and the LNA nucleosides may form base pairs according to the Watson-Crick base pairing rules. However, the “locking” of the molecule due to the methylene bridge does not allow LNA to form the ideal shape in the Watson-Crick bond. When LNA is included in a DNA or RNA oligonucleotide, the LNA may pairs with the complementary nucleotide chains more quickly and increase the stability of the double helix.

The non-cationic lipid may include one or more selected from the group consisting of a neutral lipid, anionic lipid, and cholesterol.

The neutral lipid means a lipid that is uncharged or has a neutral zwitterion form within the pH range of 4.0 to 8.0.

The neutral lipid may include any neutral lipid known to those skilled in the art. For example, the neutral lipid may include one or more selected from the group consisting of 1,2-dioleoyl-sn-glycero-3-phosphocholine (DOPC), 1-palmitoyl-2-oleoyl-sn-glycero-3-phosphoethanolamine (POPE), 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero)-3-phosphoethanolamine (DOPE), N-palmitoyl-D-erythro-sphingosylphosphorylcholine (SM), 1,2-dilauroyl-sn-glycero-3-phosphoethanolamine (DLPE), 1,2-diphytanoyl-sn-glycero-3-phosphoethanolamine (DiPPE), phosphatidyl choline, phosphatidyl ethanolamine, tetraether lipid, ceramide, sphingolipid, diacryl glycerol, and glyceride.

In another embodiment of the present disclosure, the neutral lipid is DSPC.

The anionic lipid refers to any amphipathic lipid having one or more negative charges within the range of pH 4.0 to pH 8.0.

The anionic lipid may include any anionic lipid known to those skilled in the art. For example, the anionic lipid may include one or more selected from the group consisting of dioleoylphosphatidylserine (DOPS), dimyristoylphosphatidylglycerol (DMPG), dipalmitoylphosphatidylglycerol (DPPG), diethylenetriamine pentaacetic acid (DTPA), 1,4-dipalmitoyl-tartarate-2,3-diglutaric acid (DPTGA), 1,4-disteroyl-tartarate-2,3-disuccinic acid (DSTSA), 2-carboxyheptadecanoyl heptadecylamide (CHHDA), dimyristoylphosphatidylserine (DMPS), dipalmitoylphosphatidylserine (DPPS), palmitoyloleoylphosphatidylserine (POPS), dioleoylphosphatidylglycerol (DOPG), palmitoyloleoylphosphatidylglycerol (POPG), dimyristoylphosphatidic acid (DMPA), dipalmitoylphosphatidic acid (DPPA), dioleoylphosphatidic acid (DOPA), palmitoyl-oleoylphosphatidic acid (POPA), cetyl phosphate (CetylP), and cholesterol hemisuccinate (CHEMS).

In a further embodiment of the present disclosure, the anionic lipid is DOPS.

The cholesterol provides morphological rigidity to the lipid filling within the lipid nanoparticles. The cholesterol is dispersed throughout the core and surface of the nanoparticles and plays a role in improving the stability of the nanoparticles.

The PEG-modified lipid refers to a conjugated form of lipid and PEG. The PEG-modified lipid is a lipid to which polyethylene glycol (PEG) polymer, which is a hydrophilic polymer, is bound to one end. The PEG-modified lipid within the lipid nanoparticle contributes to the particle stability of the nanoparticle in serum. The PEG-modified lipid serves as a stabilizer lipid to prevent aggregation between lipid nanoparticles. In addition, the PEG-modified lipid protects nucleic acids from degrading enzymes during in-vivo delivery of the nucleic acids, and the PEG-modified lipid enhances the stability of the nucleic acids in the body. The PEG-modified lipid may increase the half-life of the drug encapsulated in the lipid nanoparticle.

In the PEG-modified lipid, PEG may be conjugated directly to the lipid or linked to the lipid through a linker moiety. Any linker moiety suitable for linking PEG to a lipid may be used. For example, an ester-free linker moiety and an ester-containing linker moiety are included. The ester-free linker moiety includes any one selected from the group consisting of amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH2CH2C(O)—), succinamidyl (—NHC(O)CH2CH2C(O)NH—), ether, and disulfide, as well as combinations thereof (for example, a linker containing both a carbamate linker moiety and an amido linker moiety) but is not limited thereto. The ester-containing linker moiety includes any one selected from the group consisting of carbonate (—OC(O)O—), succinyl, phosphate ester (—O—(O)POH—O—), and sulfonate ester, and combinations thereof but is not limited thereto.

Any lipid capable of binding to polyethylene glycol may be used without limitation for lipid in the PEG-modified lipid. The PEG-modified lipid may be a non-cationic lipid, which is another component of lipid nanoparticles but is not limited thereto.

In a yet further embodiment of the present disclosure, the PEG-modified lipid is DSPC-PEG.

A PEG in the PEG-modified lipid is a hydrophilic polymer, and it may inhibit the adsorption of plasma proteins, thereby increasing the circulation time of lipid nanoparticles in the body and preventing aggregation between nanoparticles. In addition, the PEG-modified lipid may exhibit stealth function in vivo, preventing the degradation of nanoparticles.

The PEG may be a functionalized PEG in which a functional group is bonded to the side that is not bonded to a lipid. At this point, the functional group may include one or more selected from the group consisting of succinyl, carboxylic acid, maleimide, amine group, biotin, cyanur, and folate.

The lipid nanoparticles may have an average particle size of 10 to 2,000 nm. When the size of the lipid nanoparticle is less than 10 nm, it is not desirable since it may be difficult for the complex to be encapsulated in the lipid nanoparticle, and in that size, stability may be reduced when the lipid nanoparticle is injected into the body. In addition, even when the size exceeds 2,000 nm, it is not desirable since stability may be lowered when the composition containing the lipid nanoparticles is injected into the body.

In addition, the present disclosure provides a method of preparing a lipid nanoparticle-based drug delivery system, the method including: (a) preparing a protamine-nucleic acid complex by mixing a nucleic acid solution and a solution of a recombinant protamine protein including a solubility enhancing tag and a protamine made of an amino acid having a sequence of SEQ ID NO: 1;
(b) forming a lipid film by introducing a lipid solution including an organic solvent, a non-cationic lipid, and a PEG-modified lipid into a reactor and removing the organic solvent; and
(c) forming a lipid nanoparticle dispersion liquid by applying and dispersing the complex prepared in the step (a) onto the lipid film formed in the step (b).

Hereinafter, a method of preparing a lipid nanoparticle-based drug delivery system of the present disclosure will be described in detail step by step.

In a method of preparing a lipid nanoparticle-based drug delivery system, the step (a) involves preparing a protamine-nucleic acid complex by mixing a nucleic acid solution and a solution of a recombinant protamine protein including a solubility enhancing tag and a protamine made of an amino acid having a sequence of SEQ ID NO: 1.

Since the solubility enhancing tag, protamine, and nucleic acid are the same as described above, their description here is omitted.

When the recombinant protamine protein solution and the nucleic acid solution are mixed, the cationic recombinant protamine protein and the anionic nucleic acid may easily form a complex through electrostatic interaction.

The recombinant protamine protein may be prepared by the method below, the method including:
(1) forming a recombinant expression vector containing a gene encoding a solubility enhancing tag and a protamine gene having a base sequence of SEQ ID NO: 2;
(2) inserting the vector formed in the step (1) into a cell line and transforming the vector; and
(3) expressing recombinant protamine protein in the cell line of the step (2).

In this specification, the term “cell line” refers to a cell for expression, into which a gene or recombinant vector has been introduced to produce a target protein. The cell line may be used without limitation as long as the cell line is a cell capable of expressing glycosylated interferon lambda, and the cell line may preferably be a prokaryotic cell or a eukaryotic cell. For example, the cell line may be a CHO cell line or a mainly used HEK cell line for the expression of recombinant proteins but are not limited thereto.

In a still yet further embodiment of the present disclosure, the cell line is E. coli.

The step (3) may further include purifying the expressed recombinant protamine protein. The purifying may be used without limitation as long as the process is used for a method of purifying the expressed protein and may be performed once or more than twice.

In the method of preparing a lipid nanoparticle-based drug delivery system of the present disclosure, the step (b) involves introducing a lipid solution containing an organic solvent, a non-cationic lipid, and a PEG-modified lipid into a reactor, and removing the organic solvent to form a lipid film.

Since the non-cationic lipid and PEG-modified lipid are the same as described above, their description here is omitted.

The removing of the organic solvent may include evaporating the organic solvent in the lipid solution introduced into the reactor while maintaining the interior of the reactor in a vacuum state. At this point, only the organic solvent may be evaporated by setting the temperature in the reactor to a high temperature. Likewise, by evaporating and removing the organic solvent, the remaining lipid components excluding the organic solvent in the lipid solution form a lipid film on the inner wall and bottom of the reactor.

The type of organic solvent is not particularly limited, and any solvent that may dissolve the lipid used in the preparation of the lipid nanoparticle-based drug delivery system of the present disclosure may be used. The organic solvent may preferably be a non-polar or low-polar organic solvent. The organic solvent may include any one selected from the group consisting of benzene, butanol, butyl acetate, carbon tetrachloride, chloroform, cyclohexane, dichloroethane, dichloromethane, diethyl ether, diisopropyl ether, ethyl acetate, heptane, hexane, isooctane, methyl ethyl ketone (MEK), methyl t-butyl ether (MTBE), pentane, toluene, trichloroethylene, and xylene, and solvent mixtures thereof.

In the method of preparing a lipid nanoparticle-based drug delivery system of the present disclosure, the step (c) involves applying the complex prepared in the step (a) onto the lipid film formed in the step (b) and dispersing the complex to form a lipid nanoparticle dispersion liquid.

In the step (c), a freezing/thawing process of freezing the dispersion liquid and thawing the frozen dispersion liquid may be further included. Specifically, the freezing/thawing process means exposing the dispersion liquid to liquid nitrogen for 5 minutes and then to hot water again, and the process may be repeated 1 to 12 times. By repeating the freezing and thawing of the lipid film composition, a dispersion liquid containing lipid nanoparticles in a more uniform size may be prepared, and the drug encapsulation efficiency of lipid nanoparticles may be increased. However, when the repetition exceeds 12 times, the encapsulation efficiency of lipid nanoparticles may decrease, so repeating the process less than 12 times is preferable.

Hereinafter, the present disclosure will be described in detail by examples.

However, the following examples are merely illustrative of the present disclosure, and the content of the present disclosure is not limited by the preparation examples and examples below.

### <Example 1> Recombinant Protamine Protein Expression and Purification

First, a gene coding a S-tag and a DNA sequence (SEQ ID NO: 2) encoding a protamine were cloned into an E. coli expression plasmid pET30a to synthesize a pET30a-PA vector. Specifically, a pET30a-PA gene was synthesized on the basis of the human protamine amino acid sequence (SEQ ID NO: 1) from NCBI (#NP_002752.1). Six histidines were added to the N-termini and

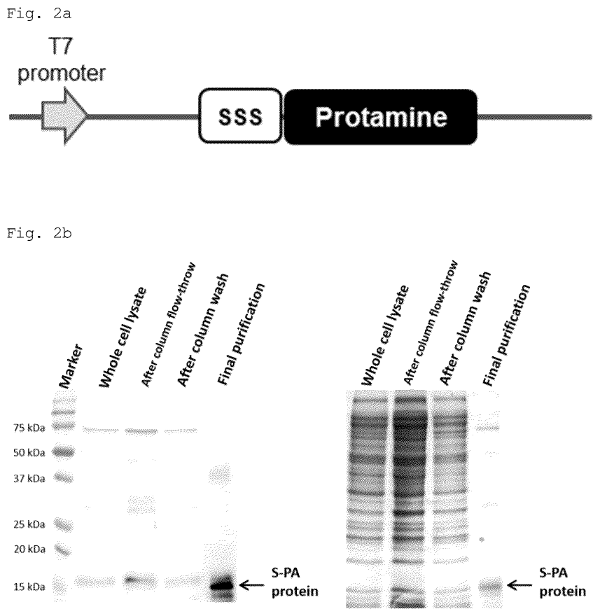

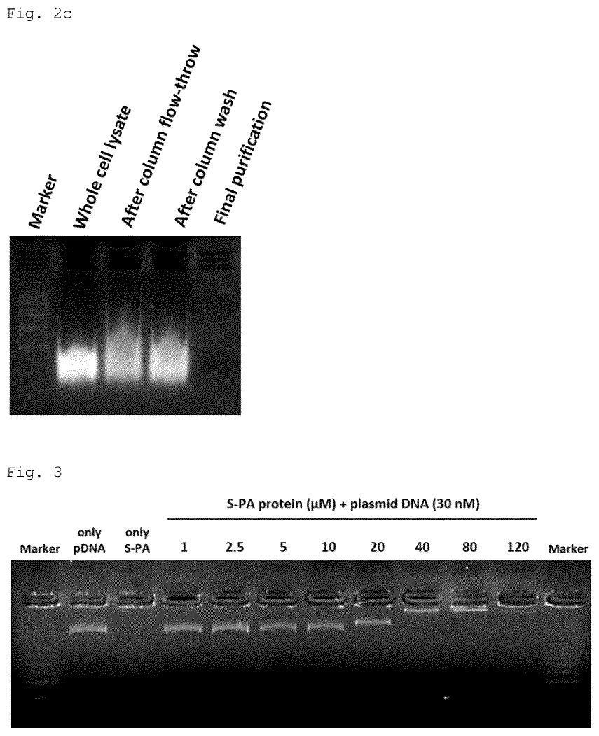

C-termini of a recombinant S-PA protein to facilitate purification after a facilitated expression in E. coli. The recombinant S-PA protein is a protein with a poly-arginine residue, but a hydrophilic sequence was added to increase the expression level of the recombinant S-PA protein in E. coli and improve the stability of a S-PA/pDNA complex (FIG. 2A).

Next, E. coli BL21 Star IM (DE3) (Thermo Fisher) was transformed with the pET30a-PA vector to express the recombinant S-PA protein. The transformed cells were cultured until the optical density at 600 nm reached 0.4 to 0.5. Afterward, the cells were induced 1 mM with isopropyl β-d-1-thiogalactopyranoside (IPTG) and cultured at 25° C. for 18 hours.

The cultured cells were lysed using a lysis buffer (100 mM Tris-HCl, 1 M NaCl, 20 mM imidazole, and 0.5 mM PMSF, pH 7.5) to purify the expressed S-PA proteins. The lysed cell solution was probe sonicated and centrifuged at 15,000 g for 20 minutes at 4° C. Then, the supernatant was reacted with HisPur™ Cobalt Resin (Thermo Fisher) and washed with washing buffer (100 mM Tris-HCl, 0.5 M NaCl, and 60 mM imidazole, pH 7.5). Thereafter, the S-PA proteins were eluted using an elution buffer (100 mM Tris-HCl, 0.3 M NaCl, and 300 mM imidazole, pH 7.5). The eluted proteins were further purified in a Strong Cation Exchange Column (Thermo Fisher) (FIG. 2B). It was confirmed that there was no E. coli RNA contamination during the purification process (FIG. 2C). Afterward, the purified proteins were dialyzed using a storage buffer (100 mM Tris-HCl, 0.2 M NaCl, 0.1 mM EDTA, 0.5 mM PMSF and 20% glycerol, pH 7.5) and stored at −80° C.

### <Experimental Example 1> Confirmation of Binding of Recombinant Protamine Protein (S-PA) and Plasmid DNA (pDNA) and Formation of S-PA/pDNA Complexes

The recombinant protamine proteins (S-PA) purified in Example 1 were prepared to have concentrations (1, 2.5, 5, 10, 20, 40, 80, and 120 μM). The recombinant protamine proteins (S-PA) of each concentration were reacted with 30 nM plasmid DNA (pDNA) in 1 mL of phosphate-buffered saline (PBS, pH 7.2) to form S-PA/pDNA complexes. The plasmid DNA (#HG18546-UT, Sinobiological) used here encoded an HIC1 gene. Next, agarose gel electrophoresis analysis was performed to confirm the optimized concentration of the formed complexes.

As a result of electrophoresis analysis, it was confirmed that complexes were formed through the binding between 30 nM plasmid DNA and the recombinant protamine proteins when the concentration of recombinant protamine proteins (S-PA) was 20 UM or more (FIG. 3). Therefore, it was confirmed that complexes were efficiently formed through the binding between 30 nM plasmid DNA and recombinant protamine proteins when the concentration of the S-PAs was 30 μM (FIG. 3).

### <Experimental Example 2> Stability Assessment of S-PA/pDNA Complexes

To assess the stability of the S-PA/pDNA complexes, S-PA/pDNA complexes were prepared by inducing a reaction to cause binding between 30 μM of the S-PA purified in Example 1 and five types of pDNA, which was at different concentrations (0, 7.5, 15, 30, and 60 nM), by electrostatic interaction in PBS (pH 7.2) at room temperature. As a control, 30 μM of protamine sulfate (PS) was reacted with pDNA of its respective concentration of 0, 7.5, 15, 30, and 60 nM under the same conditions as above to prepare PS/pDNA complexes. Next, the stability of the prepared complexes was assessed using a UV-visible spectrometer and with the naked eye.

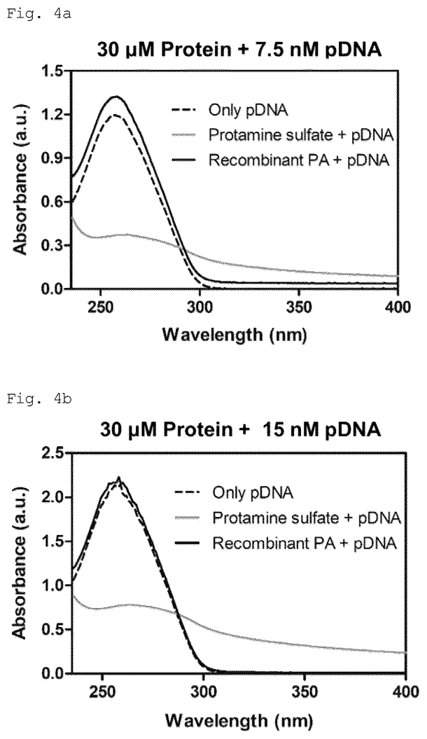

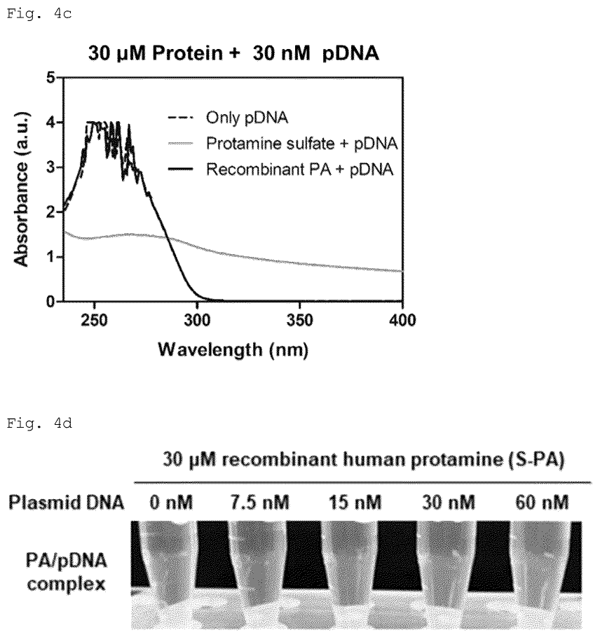

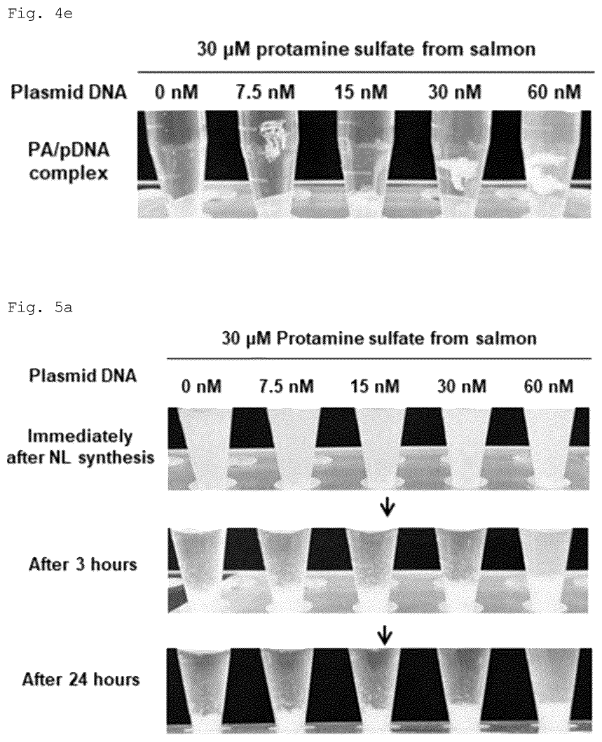

Absorbance measurement results using a UV-visible spectrometer showed that when the S-PA/pDNA complexes were reacted with various concentrations of plasmid, the peak at 280 nm was measured similarly to the absorbance result measuring only the plasmid, thereby a similar level of stability was observed when only the plasmid was present. On the other hand, the PS/pDNA complexes were confirmed to have relatively low binding stability because the peak at the corresponding wavelength was measured to be relatively low (FIGS. 4A to 4C).

In addition, when each sample was observed with the naked eye, no aggregation was observed at any concentration in the case of the S-PA/pDNA complexes. On the other hand, it was confirmed that aggregation occurred in the PS/pDNA complexes serving as the control under all concentration conditions except pDNA 0 nM (FIGS. 4D and 4E). Through these results, it was confirmed that the S-PA had superior binding stability to PS when forming a complex with a plasmid DNA.

### <Experimental Example 3> Stability Assessment of Lipid Nanoparticles Encapsulating S-PA/pDNA Complexes

To confirm the dispersion stability of lipid nanoparticles encapsulating S-PA/pDNA complexes, hydration was performed with a lipid film made of distearoylphosphatidylcholine (DSPC) and cholesterol by using the S-PA/pDNA complexes prepared in Experimental Example 2, and lipid nanoparticles (LNPs) were synthesized. The lipid nanoparticles were prepared through a thin film hydration method. Briefly, DSPC:DSPC-PEG:cholesterol (mol %=58:2:40) was dissolved in 1 mL of chloroform, and the solvent was evaporated to form lipid films. Next, the formed lipid films were hydrated with 1 ml of S-PA/pDNA complexes. Then, after a freeze-thaw procedure, the LNP solution was purified using a CL4B size extraction column. Afterward, by filtering the purified LNP solution through a 0.2 μm syringe filter, lipid nanoparticles encapsulating S-PA/pDNA complexes were prepared. As a control, the PS/pDNA complexes prepared in Experimental Example 2 were used. Lipid nanoparticles encapsulating PS/pDNA complexes were prepared in the same manner as above. Immediately after the synthesis, 3 hours later (stored at 4° C.), and 24 hours later (stored at 4° C.), the stability of the synthesized lipid nanoparticle sample was assessed through dynamic light scattering (DLS) analysis capable of measuring particles with the naked eye and sizes ranging from 0.3 nm to 10 μm.

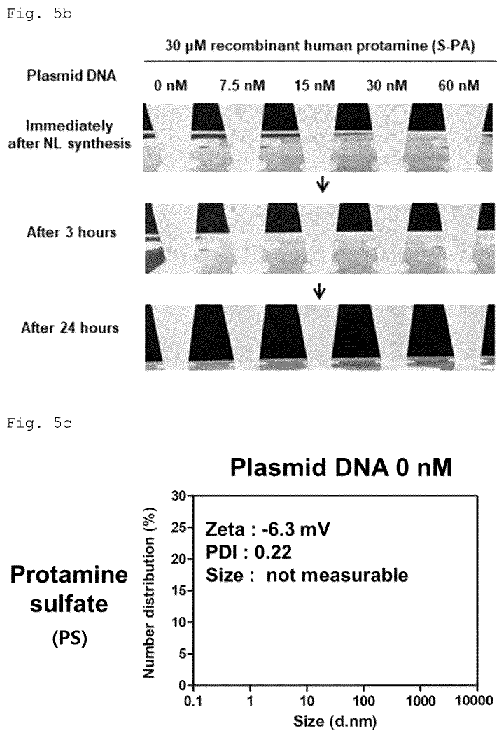

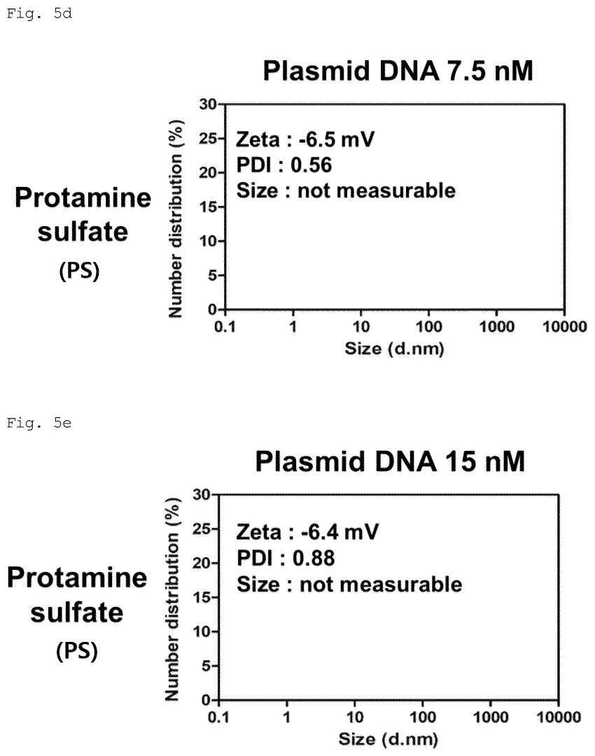

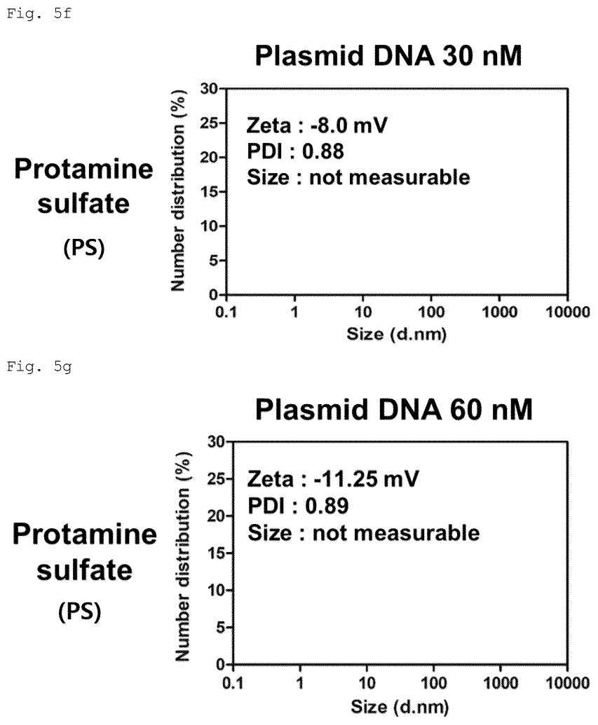

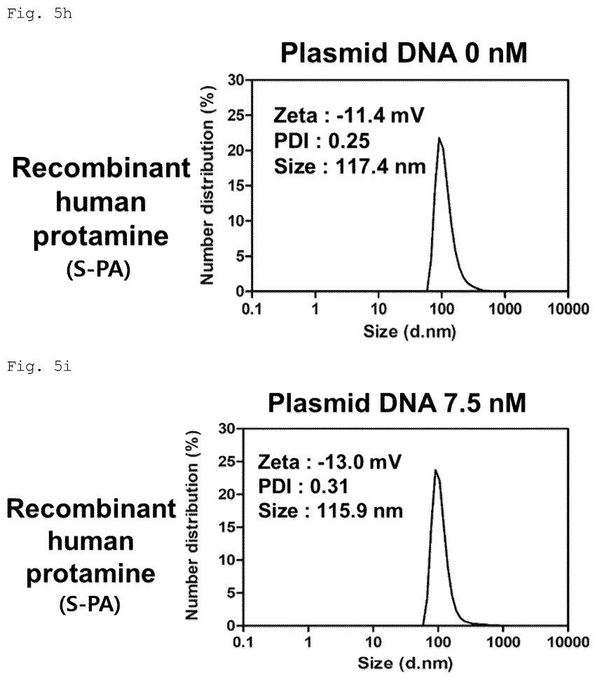

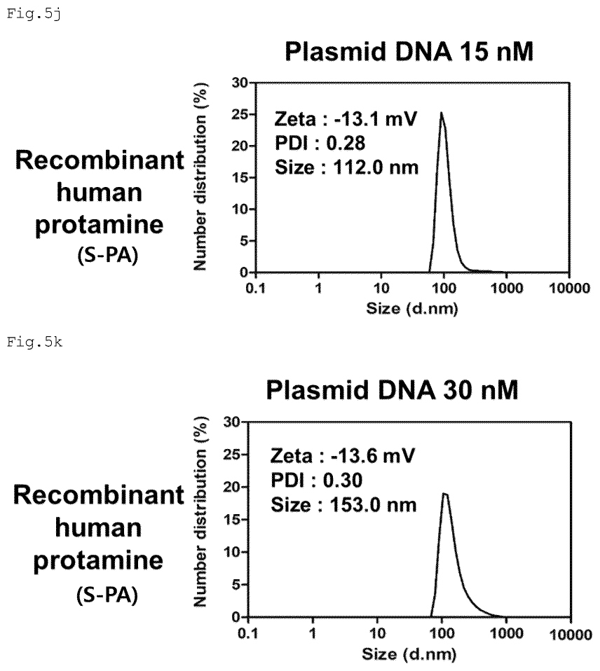

As a result of visual observation of each sample, it was confirmed that in the case of the synthesized lipid nanoparticles encapsulating PS/pDNA complexes, most particles aggregated and settled after 3 hours of storage (FIG. 5A). On the other hand, in the case of the synthesized lipid nanoparticles encapsulating the S-PA/pDNA complexes, particles were not found to be aggregated and settled even after 24 hours of storage at 4° C. (FIG. 5B).

Next, as a result of DLS analysis, the synthesized lipid nanoparticles encapsulating PS/pDNA complexes had a polydispersity index (PDI) of 0.5 or more at all concentrations except pDNA 0 nM, and the particle size was outside the measurable range. Thus, it was confirmed that dispersion stability was poor (FIGS. 5C to 5G). On the other hand, the synthesized lipid nanoparticles encapsulating S-PA/pDNA complexes had a PDI of 0.2 to 0.3 in all samples, and the particle size was an average of 110 to 150 nm (FIGS. 5H to 5L). These results confirmed that the synthesized lipid nanoparticles encapsulating the S-PA/pDNA complexes had excellent storage stability.

### <Experimental Example 4> Confirmation of Gene Expression Level within Cells after Delivering Synthesized Lipid Nanoparticles to Cells

To confirm the expression level of genes delivered to cells by the lipid nanoparticles of the present disclosure, lipid nanoparticles were synthesized in the same manner as in Experimental Example 3 above using 30 μM S-PA protein and 30.82 nM plasmid DNA. MDA-MB-468 cells were treated with the lipid nanoparticles at a ratio of 2.48×10¹¹±2.75×10¹⁰ LNP particles/1.3×10⁶ cells. After 24 hours, the cells were collected, and the protein expression level was confirmed by Western blotting. At this point, similarly, plasmid DNA (pCMV-HIC1, #HG18546-UT, Sinobiological) encoding the HIC1 (Human hypermethylated in cancer 1, NCBI number: NM_006497.3) gene was used.

Specifically, MDA-MB-468 cells used in the experiment were cultured at 37° C. under a 5% CO₂ environment in ATCC modified RPMI medium (Gibco) supplemented with 10% fetal bovine serum (FBS) and 1% penicillin/streptomycin. The MDA-MB-468 cells were inoculated into 60 mm 2-cell culture plates (1.3×10⁶ cells/plate) 24 hours before LNP treatment, and then the MDA-MB-468 cells were treated with LNPs (S-PA/pDNA) (2.48×10¹¹±2.75×10¹⁰ LNP particles/2.5 mL opti-MEM) and cultured at 37° C. for 24 hours.

The LNP-treated MDA-MB-468 cells were lysed in RIPA buffer containing protease inhibitor and phosphatase inhibitor (Sigma). Total cell lysates were quantified using the BCA protein assay kit (Thermo Scientific). Then, the cell lysates (50 μg) were loaded on a 4% to 15% gradient SDS-PAGE gel (Biorad) under denaturing conditions and transferred to a PVDF membrane (iBlot2 Dry Blotting System, Thermo Scientific). The membrane was blocked with 5% skim milk for 1 hour at room temperature. The cell lysates were reacted with primary antibodies for HIC1 (Proteintech) and actin (Santa Cruz) overnight at 4° C. Afterward, the resulting product was reacted with HRP-conjugated secondary antibodies at room temperature for 2 hours, and the membrane was subjected to Western ECL treatment. Luminescence images were analyzed using LAS500 (GE Healthcare).

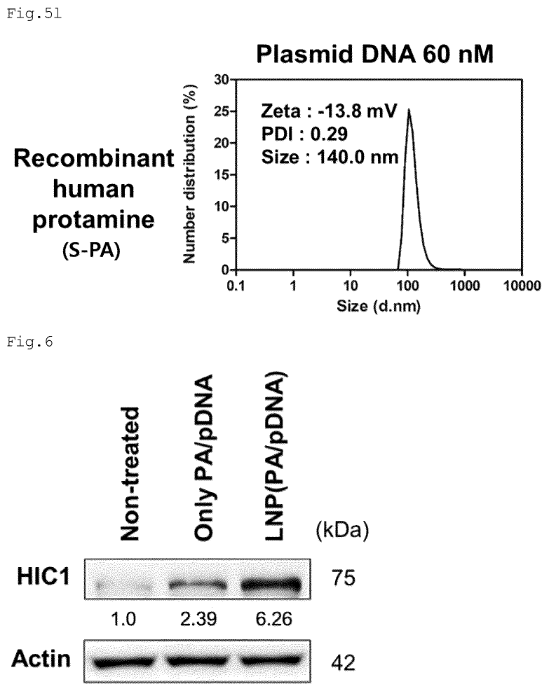

As a result of Western blotting, it was confirmed that the expression level of HIC1 protein in the experimental group treated with lipid nanoparticles encapsulating the S-PA/pDNA complexes was 6.26 times higher than in the non-treated group. When compared to the experimental group treated with only the S-PA/pDNA complexes, it was confirmed that the expression level of HIC1 protein increased 2.62-fold in the experimental group treated with lipid nanoparticles encapsulating the S-PA/pDNA complexes (FIG. 6).

The results demonstrated that lipid nanoparticles encapsulating the S-PA/pDNA complexes could be a carrier for effectively delivering plasmid DNA to cells.

The present disclosure has been described through specific details and limited examples as described above. However, this is only provided to aid the overall understanding of the present disclosure, and the present disclosure is not limited to the above examples. Those skilled in the art can make various modifications and variations from this description.

Therefore, the spirit of the present disclosure should not be limited to the described examples. Not only the scope of the patent claims described later but also all things that are equivalent or equivalent to the scope of this patent claim fall within the scope of the present disclosure.

1. A lipid nanoparticle-based drug delivery system comprising:
(a) a protamine/nucleic acid complex of a recombinant protamine protein and a nucleic acid, the recombinant protamine protein comprising a solubility enhancing tag and a protamine made of an amino acid having a sequence of SEQ ID NO: 1;
(b) a non-cationic lipid; and
(c) a PEG-modified lipid.

2. The drug delivery system of claim 1, wherein the PEG-modified lipid is a combination of the non-cationic lipid and a polyethylene glycol (PEG).

3. The drug delivery system of claim 1, wherein the solubility enhancing tag is bound to the N-terminus or C-terminus of the protamine or bound to both the N-terminus and C-terminus of the protamine.

4. The drug delivery system of claim 1, wherein the solubility enhancing tag comprises any one selected from the group consisting of S-tag, b′a′ domain of PDI (PDIb′a′), protein disulfide isomerase, maltose binding protein (MBP), hexahistidine (His6), thioredoxin (Trx), glutathione S-transferase (GST), N-utilization substance Protein A (NusA), small ubiquitin related modifier (SUMO), and protein disulfide isomerase (PDI).

5. The drug delivery system of claim 1, wherein the nucleic acid comprises one or more selected from the group consisting of a plasmid DNA, a single-stranded DNA, a double-stranded DNA, a miRNA, a siRNA, a ssRNA, a shRNA, an mRNA, an ncRNA, an antisense RNA, and an LNA.

6. The drug delivery system of claim 1, wherein the non-cationic lipid comprises one or more selected from the group consisting of a neutral lipid, an anionic lipid, and a cholesterol.

7. The drug delivery system of claim 6, wherein the neutral lipid comprises one or more selected from the group consisting of 1,2-dioleoyl-sn-glycero-3-phosphocholine (DOPC), 1-palmitoyl-2-oleoyl-sn-glycero-3-phosphoethanolamine (POPE), 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero)-3-phosphoethanolamine (DOPE), N-palmitoyl-D-erythro-sphingosylphosphorylcholine (SM), 1,2-dilauroyl-sn-glycero-3-phosphoethanolamine (DLPE), 1,2-diphytanoyl-sn-glycero-3-phosphoethanolamine (DiPPE), phosphatidyl choline, phosphatidyl ethanolamine, tetraether lipid, ceramide, sphingolipid, diacryl glycerol, and glyceride.

8. The drug delivery system of claim 6, wherein the anionic lipid comprises one or more selected from the group consisting of dioleoylphosphatidylserine (DOPS), dimyristoylphosphatidylglycerol (DMPG), dipalmitoylphosphatidylglycerol (DPPG), diethylenetriamine pentaacetic acid (DTPA), 1,4-dipalmitoyl-tartarate-2,3-diglutaric acid (DPTGA), 1,4-disteroyl-tartarate-2,3-disuccinic acid (DSTSA), 2-carboxyheptadecanoyl heptadecylamide (CHHDA), dimyristoylphosphatidylserine (DMPS), dipalmitoylphosphatidylserine (DPPS), palmitoyloleoylphosphatidylserine (POPS), dioleoylphosphatidylglycerol (DOPG), palmitoyloleoylphosphatidylglycerol (POPG), dimyristoylphosphatidic acid (DMPA), dipalmitoylphosphatidic acid (DPPA), dioleoylphosphatidic acid (DOPA), palmitoyl-oleoylphosphatidic acid (POPA), cetyl phosphate (CetylP), and cholesterol hemisuccinate (CHEMS).

9. The drug delivery system of claim 1, wherein the lipid nanoparticles have an average particle size of 10 to 2,000 nm.

10. A method of preparing a lipid nanoparticle-based drug delivery system, the method comprising:
(a) preparing a protamine-nucleic acid complex by mixing a nucleic acid solution and a solution of a recombinant protamine protein comprising a solubility enhancing tag and a protamine made of the amino acid having a sequence of SEQ ID NO: 1;
(b) forming a lipid film by introducing a lipid solution comprising an organic solvent, a non-cationic lipid, and a PEG-modified lipid into a reactor and removing the organic solvent; and
(c) forming a lipid nanoparticle dispersion liquid by applying and dispersing the complex prepared in the step (a) onto the lipid film formed in the step (b).

11. The method of claim 10, wherein the PEG-modified lipid is a combination of the non-cationic lipid and a polyethylene glycol (PEG).

12. The method of claim 10, wherein the solubility enhancing tag is bound to the N-terminus or C-terminus of the protamine, or both the N-terminus and C-terminus of the protamine.

13. The method of claim 10, wherein the solubility enhancing tag comprises any one selected from the group consisting of S-tag, b′a′ domain of PDI (PDIb′a′), protein disulfide isomerase, maltose binding protein (MBP), hexahistidine (His6), thioredoxin (Trx), glutathione S-transferase (GST), N-utilization substance Protein A (NusA), small ubiquitin related modifier (SUMO), and protein disulfide isomerase (PDI).

14. The method of claim 10, wherein the nucleic acid comprises one or more selected from the group consisting of a plasmid DNA, a single-stranded DNA, a double-stranded DNA, a miRNA, a siRNA, a ssRNA, a shRNA, an mRNA, an ncRNA, an antisense RNA, and an LNA.

15. The method of claim 10, wherein the non-cationic lipid comprises one or more selected from the group consisting of a neutral lipid, an anionic lipid, and a cholesterol.

16. The method of claim 15, wherein the neutral lipid comprises one or more selected from the group consisting of 1,2-dioleoyl-sn-glycero-3-phosphocholine (DOPC), 1-palmitoyl-2-oleoyl-sn-glycero-3-phosphoethanolamine (POPE), 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero)-3-phosphoethanolamine (DOPE), N-palmitoyl-D-erythro-sphingosylphosphorylcholine (SM), 1,2-dilauroyl-sn-glycero-3-phosphoethanolamine (DLPE), 1,2-diphytanoyl-sn-glycero-3-phosphoethanolamine (DiPPE), phosphatidyl choline, phosphatidyl ethanolamine, tetraether lipid, ceramide, sphingolipid, diacryl glycerol, and glyceride.

17. The method of claim 15, wherein the anionic lipid comprises one or more selected from the group consisting of dioleoylphosphatidylserine (DOPS), dimyristoylphosphatidylglycerol (DMPG), dipalmitoylphosphatidylglycerol (DPPG), diethylenetriamine pentaacetic acid (DTPA), 1,4-dipalmitoyl-tartarate-2,3-diglutaric acid (DPTGA), 1,4-disteroyl-tartarate-2,3-disuccinic acid (DSTSA), 2-carboxyheptadecanoyl heptadecylamide (CHHDA), dimyristoylphosphatidylserine (DMPS), dipalmitoylphosphatidylserine (DPPS), palmitoyloleoylphosphatidylserine (POPS), dioleoylphosphatidylglycerol (DOPG), palmitoyloleoylphosphatidylglycerol (POPG), dimyristoylphosphatidic acid (DMPA), dipalmitoylphosphatidic acid (DPPA), dioleoylphosphatidic acid (DOPA), palmitoyl-oleoylphosphatidic acid (POPA), cetyl phosphate (CetylP), and cholesterol hemisuccinate (CHEMS).

18. The method of claim 10, wherein the step (c) involves repeating a freezing/thawing process 1 to 12 times, the process comprising freezing a dispersion liquid and thawing the frozen dispersion liquid.

19. The method of claim 10, wherein the recombinant protamine protein is prepared by the method below, the method comprising:
(1) forming a recombinant expression vector containing a gene encoding a solubility enhancing tag and a protamine gene having a base sequence of SEQ ID NO: 2;
(2) inserting the vector formed in the step (1) into a cell line and transforming the vector; and
(3) expressing a recombinant protamine protein in the cell line of the step (2).

20. The method of claim 19, wherein the step (3) further involves purifying the expressed recombinant protamine protein.


SEQUENCE LISTING (3 sequences)

SEQ ID NO: 1 - protein, 50 aa, synthetic construct
  REGION 1..50: note = protamine(PRM1)
ARYRCCRSQSRSRYYRQRQRSRRRRRRSCQTRRRAMRCCRPRYRPRCRRH

SEQ ID NO: 2 - DNA, 153 bp, synthetic construct
  misc_feature 1..153: note = protamine(PRM1)
  source 1..153: mol_type = other DNA
atggcgcgttatcgttgctgccgtagccaaagccgtagccgttattatcgtcagcgtcagcgtagccgtcgtcgtcgtcgtcgtagctgccagacccgccgccgtgcgatgcgttgctgccgtccgcgttaccgcccgcgttgccgtcgtcac

SEQ ID NO: 3 - protein, 65 aa, synthetic construct
  REGION 1..65: note = s-tagged protamine(PRM1)
KETAAAKFERQHMDSARYRCCRSQSRSRYYRQRQRSRRRRRRSCQTRRRAMRCCRPRYRPRCRRH